(19) United States
(12) Patent Application Publication
(10) Pub. No.: US 2016/0367606 A1
(43) Pub. Date: Dec. 22, 2016

(54) COMPOSITION HAVING HYDROLYZED COLLAGEN AND MANUKA HONEY

(71) Applicant: GEORGE D. PETITO, BETHLEHEM, PA (US)

(72) Inventor: GEORGE D. PETITO, BETHLEHEM, PA (US)

(21) Appl. No.: 15/179,913

(22) Filed: Jun. 10, 2016

Related U.S. Application Data
(60) Provisional application No. 62/180,583, filed on Jun. 16, 2015.

(51) Int. Cl. A61K 35/644; A61K 9/00; A61K 31/11; A61K 47/42

(52) U.S. Cl. CPC A61K 35/644; A61K 47/42; A61K 9/0014; A61K 31/11

(57) ABSTRACT

The composition having hydrolyzed collagen and Manuka honey includes hydrolyzed collagen in an amount ranging from between 5% to 85% by weight and Manuka honey in an amount ranging from between 5% to 85% by weight in water. The Manuka honey can have a methylglyoxal concentration greater than 100 mg/kg; and preferably greater than 550 mg/kg.

### CROSS-REFERENCE TO RELATED APPLICATIONS

This application claims the benefit of U.S. Provisional Patent Application Ser. No. 62/180,583, filed Jun. 16, 2015.

### BACKGROUND OF THE INVENTION

1. Field of the Invention

The present invention relates to medical compositions, and particularly, to a composition having hydrolyzed collagen and Manuka honey.

2. Description of the Related Art

Honey, made from the nectar of the Manuka tree (tea tree), has exceptional phytochemical-derived, antimicrobial properties, e.g., antibacterial, antifungal, and/or anti-viral properties. In 2008, it was discovered that Manuka honey contains methylglyoxal, and that methylglyoxal gives rise to the antimicrobial properties of Manuka honey. Methylglyoxal has more advantageous properties than the hydrogen peroxide produced in all raw honey from glucose oxidase, which comes from bees during honey production. For example, dietary methylglyoxal is resistant to heat, light, and enzymatic activity, while glucose oxidase/hydrogen peroxide activity is destroyed by heat. Other food items known to contain significant amounts of dietary methylglyoxal are coffee and cocoa (approx. 100 mg/kg). The concentrations of methylglyoxal in coffee and cocoa, however, are small compared to the levels in some Manuka honeys.

Methylglyoxal is found in all honeys in very small concentrations. Manuka honey methylglyoxal contents can range from 0-1000 mg/kg. Anything higher than 100 mg/kg is considered antibacterial, with higher concentrations of methylglyoxal being associated with greater antibacterial activity. A methylglyoxal concentration greater than 400 mg/kg has been proven to kill a large number of powerful bacteria and viruses immune to other classic antibiotics, such as: Helicobacter pylori—the bacterium known to cause many stomach and duodenal ulcers; Staphylococcus aureus or MRSA—a super-bug with high levels of antibiotic resistance; Escherichia coli—known to cause serious food poisoning; and Streptococcus pyogenes—causes sore throats. Although it is clear that the dietary methylglyoxal present in many Manuka honeys has strong anti-bacterial properties, its efficacy and safety when ingested is often hotly debated.

Thus, a composition having hydrolyzed collagen and Manuka honey solving the aforementioned problems is desired.

### SUMMARY OF THE INVENTION

The composition having hydrolyzed collagen and Manuka honey includes hydrolyzed collagen in an amount ranging from between 5% to 85% by weight and Manuka honey in an amount ranging from between 5% to 85% by weight in water. The Manuka honey can have a methylglyoxal concentration greater than 100 mg/kg, and preferably greater than 550 mg/kg.

These and other features of the present invention will become readily apparent upon further review of the following specification.

### DETAILED DESCRIPTION OF THE PREFERRED EMBODIMENTS

A composition having hydrolyzed collagen and Manuka honey includes about 5% to about 85% hydrolyzed collagen by weight and about 5% to about 85% Manuka honey by weight in water. The composition can be used topically, parenterally, or delivered as either an orally ingestible liquid, tablet or capsule for wound healing, and particularly for the healing of surgical wounds.

The hydrolyzed collagen can be any suitable type of hydrolyzed collagen having a molecular weight (“MW”) ranging from 500 MW to 10,000 MW. Lower MW hydrolyzed collagen can have more cross-linking action with honey than higher MW hydrolyzed collagen. As such, commercially available hydrolyzed collagen having the lowest MW is preferred. The hydrolyzed collagen can be derived from any fish, porcine, bovine, fermentation, or vegetable source. Preferably, the hydrolyzed collagen is derived from a bovine source.

The Manuka honey can be any suitable type of Manuka honey, such as preserved Manuka honey. The Manuka honey can have a methylglyoxal concentration greater than 100 mg/kg, and preferably greater than 550 mg/kg.

The composition can take the physical form used in topical administration, e.g., gel, spray, powder, paste, foam, film, and incorporation in a dressing bandage, a topically applied patch, or in internal administration, e.g., an injectable liquid or orally ingestible liquid.

The composition can be administered to a patient to facilitate wound healing. The Manuka honey in the composition can facilitate control of the bacterial load of a wound and allow the wound bed to have properties similar to those in the extracellular matrix. For example, the methylglyoxal in the Manuka honey can provide antimicrobial properties. The glucose present in the honey is converted to sodium hyaluronate (HA) in a wound environment. HA is a viscoelastic substance consisting of disaccharide chains from glucuronic acid and N-acetylglucosamine. As described below, HA interacts with the hydrolyzed collagen in the extracellular matrix to create an environment conducive to wound healing and, more specifically, to scar-free healing (similar to fetal healing).

The amino groups of the hydrolyzed collagen crosslink with the methylglyoxal in the honey. This cross-linking can allow the hydrolyzed collagen to be time released. For example, availability to the cell site can be demonstrably longer and more controlled than would be if the hydrolyzed collagen is used alone. This cross-linking can also allow immobilization of enzymes and cross-linking with polysaccharides of the HA to produce high density gels and films. In particular, incorporation of saccharides into newly formed collagen is achieved by the cross-linking action of the mono and disaccharides of HA with the hydrolyzed collagen.

It is believed that the combination of collagen and honey increases cellular activity due to increased growth factor and cytokine activity through acid activation (acidic pH produced through growth factors such as TGF-B). It is further believed that hydrolyzed collagen offers many advantages to the composition. For example, hydrolyzed collagen has many more active chemical sites than native collagen and affords more control by virtue of its molecular weight. The use of hydrolyzed collagen can be imperative for achieving the cross-linking described above, which limits the reticular (net-like) pattern to the extent necessary for both wound healing and scar reduction. In contrast, collagen (Type 1) occurs only in “parallel,” and not mesh-like, filaments, and cross-linking is severely inhibited.

The composition can be prepared in any suitable manner, and is not limited to the method described herein. Hydrolyzed collagen is transformed from the powder state to a highly viscous gel, similar to the “as conceived” honey. The initial stages involve heating injection grade water to approximately 135-145° F., and adding the collagen powder while stirring at approximately 35-45 rpm. Once the desired viscosity is achieved (visual and with activity level by formulary), the honey is added at 130° F. or less, but not less than 115° F. It must be noted that viscosity is dependent on the molecular weight of the hydrolyzed collagen and the amount of honey added. It is imperative that the temperature be kept constant to achieve a final product that meets both viscosity and antimicrobial specification(s). The amount of hydrolyzed collagen can vary from between 5% to 95% by weight and the amount of Manuka honey can vary from between 5% to 95% by weight. The water injected into the composition can include varying degrees of crosslinking and antibacterial effectiveness. The final product(s) can be aseptically filled or post sterilized, but not required.

The presence of Manuka honey in the composition confers a number of additional benefits. The Manuka honey acts like a plasticizer, impacting the drying rate and conforming rate of the resulting collagen gel. Manuka honey also assists in blood clotting.

It is to be understood that the present invention is not limited to the embodiments described above, but encompasses any and all embodiments within the scope of the following claims.

1. A composition having hydrolyzed collagen and Manuka honey, the composition comprising:
between 5% and 95% hydrolyzed collagen by weight; and
between 5% and 95% Manuka honey by weight, the balance being water.

2. The composition according to claim 1, wherein the composition is formulated for topical use.

3. The composition according to claim 1, wherein the composition is formulated for at least one of parenteral administration and oral administration.

4. The composition according to claim 1, wherein said Manuka honey has a concentration of methylglyoxal of at least 100 mg/kg.

5. The composition according to claim 1, wherein said Manuka honey has a concentration of methylglyoxal of at least 550 mg/kg.

6. The composition according to claim 1, wherein said hydrolyzed collagen is low molecular weight hydrolyzed collagen.

7. A method of making a composition having hydrolyzed collagen and Manuka honey, the method comprising the steps of:
heating water to a temperature ranging from between 135° F. to 145° F.;
mixing the heated water with collagen powder to form a viscous gel; and
adding the Manuka honey to the viscous gel to form a homogenized mixture,
wherein the hydrolyzed collagen ranges from between 5% to 95% by weight and the Manuka honey ranges from between 5% to 95% by weight.

8. The method of making a composition having hydrolyzed collagen and Manuka honey according to claim 7, wherein said Manuka honey has a concentration of methylglyoxal of at least 100 mg/kg.

9. The method of making a composition having hydrolyzed collagen and Manuka honey according to claim 7, wherein said Manuka honey has a concentration of methylglyoxal of at least 550 mg/kg.

10. The method of making a composition having hydrolyzed collagen and Manuka honey according to claim 7, wherein said hydrolyzed collagen is low molecular weight hydrolyzed collagen.

11. A method of treating a wound comprising administering to the site of a wound a composition having between 5% and 95% hydrolyzed collagen by weight and having between 5% and 95% Manuka honey by weight.

12. The method of treating a wound according to claim 11, wherein said Manuka honey has a concentration of methylglyoxal of at least 100 mg/kg.

13. The method of treating a wound according to claim 11, wherein said Manuka honey has a concentration of methylglyoxal of at least 550 mg/kg.

14. The method of treating a wound according to claim 11, wherein said hydrolyzed collagen is low molecular weight hydrolyzed collagen.
